(19) United States
(12) Patent Application Publication
(10) Pub. No.: US 2023/0058083 A1
(43) Pub. Date: Feb. 23, 2023

(54) SYSTEM AND METHOD FOR REDUCING A KINETOSIS EFFECT IN AT LEAST ONE PASSENGER OF AN VEHICLE

(71) Applicant: ZF Friedrichshafen AG, Friedrichshafen (DE)

(72) Inventors: Mohamad Alayan, Tettnang (DE); Florian Nicola, Merzig (DE); Istvan Hegedüs-Bite, Eriskirch (DE); Ilker Bagci, Ravensburg (DE); Hubert Bichelmeier, Tettnang (DE)

(73) Assignee: ZF Friedrichshafen AG, Friedrichshafen (DE)

(21) Appl. No.: 17/760,251

(22) PCT Filed: Feb. 3, 2021
(86) PCT No.: PCT/EP2021/052543

§ 371 Date: Aug. 5, 2022

(30) Foreign Application Priority Data
Feb. 6, 2020 (DE) 10 2020 201 435.3

(51) Int. Cl. A61M 21/00; H04R 1/10

(52) U.S. Cl. CPC A61M 21/00; H04R 1/1008; H04R 1/1041; A61M 2021/0027; A61M 2230/50; A61M 2230/63; A61M 2230/005; A61M 2210/0662; A61M 2205/3303; A61M 2205/3561; B60R 16/037

(57) ABSTRACT

A system for reducing a kinetosis effect in at least one passenger of a vehicle comprises at least one set of headphones having two loudspeakers and at least one first ear bud, wherein the at least one set of headphones is designed, at least by means of a sensor device integrated in the first ear bud, to detect the kinetosis effect in the auditory canal of the respective passenger via measurement of the body temperature and to provide acoustic signals by means of the two loudspeakers in order to reduce the kinetosis effect of the passenger depending on the kinetosis effect detected. Forces acting on the passenger and their countenance can also be measured.

The invention relates to a system and a method for reducing a kinetosis effect in at least one passenger of a vehicle. A vehicle is to be understood as a means of transportation in the broadest sense. The vehicle can therefore be designed as a gondola.

Kinetosis, also called “motion sickness”, can occur during journeys in different means of transportation, e.g., motor vehicles, aircraft, or ships. In particular, a kinetosis effect often occurs when the passengers are located in an interior of the means of transportation without visual contact with the real horizon or direct their view on a resting point within the means of transportation. The sensitivity to kinetosis effects can be dependent, for example, on the position of the passenger in the means of transportation and on their age, size, or weight. In the transportation situations triggering kinetosis, contradictory stimuli are sent to the brain of the affected persons by the so-called sense of sight, on the one hand, and from the sense of equilibrium, on the other hand, which result there in contradictory perceptions with respect to orientation, movement, and acceleration of one's own body. The sense of sight is to be understood as a capability of orienting oneself in space with the aid of the stimuli originating from the visual apparatus. In contrast, the sense of equilibrium is used to perceive the orientation of one's own body in space and the accelerations acting on the body.

A situation triggering kinetosis or kinetosis effects is, for example, reading or using a mobile terminal, for example, a smart phone or tablet, during the driving operation of a motor vehicle. The sense of sight reports substantial immobility of one's own body relative to the surroundings here, while the sense of equilibrium picks up the accelerations changing more or less continuously, to which the passenger is really subjected, and triggers the corresponding perception of a movement of the body, with changing velocities and in changing directions, in the brain. Such discrepancies of the perceptions of movement, orientation, and acceleration can then result in the onset of kinetosis, which can be expressed in the form of headache, dizziness, increase in pulse rate, and nausea up to vomiting.

For example, DE 10 2014 019 579 A1 discloses a system for operating a display unit in a vehicle. The display unit can be designed as data glasses and can make a text visible to an occupant of the vehicle wearing the data glasses for reading. The vehicle has a plurality of sensors for detecting items of movement information of the vehicle. The display unit is configured to receive the items of movement information and to display a discreet background for the text which is calculated in real-time consistently with the accelerations of the vehicle in dependence on the received items of movement information. In this way, risk of kinetosis is reduced for the wearer of the data glasses while reading the text.

The present invention is based on the object, proceeding from the prior art, of providing an alternative system and a method for reducing a kinetosis effect for at least one passenger of a vehicle. The object is achieved by the subject matter of claims 1 and 13. Further advantageous embodiments can be inferred from the dependent claims.

The invention relates to a system for reducing a kinetosis effect in at least one passenger of a vehicle, comprising at least one set of headphones having two loudspeakers and having at least one first earplug, wherein the at least one set of headphones is configured to detect the kinetosis effect in the auditory canal of the respective passenger at least by means of a sensor device integrated in the first earplug and to provide acoustic signals for reducing the kinetosis effect of the respective passenger by means of the two loudspeakers in dependence on the detected kinetosis effect.

Headphones are to be understood as an electronic component which a passenger, thus the user or wearer of the headphones, positions and fixes on the head, in particular on the ears. The headphones can be designed in the type “over-ear”, “on-ear”, “in-ear”, or “earbuds”. Over-ear headphones have a headband, come into contact on the head, and are formed enclosing the ear, wherein on-ear headphones have a headband and come into contact on the pinna, wherein furthermore in-ear headphones are arranged in the auditory canal, and wherein earbud headphones come into contact in the center of the pinna. The headphones are configured to be worn by a user, thus by the respective passenger, to play back items of information or entertainment media, for example music, via the two loudspeakers during driving operation of the vehicle. Loudspeakers are to be understood not only as airborne-sound loudspeakers, but also bone conduction loudspeakers, which transmit vibrations to the pinna directly via the bone in order to play back music and acoustic signals to reduce the kinetosis effects.

In particular, the system according to the invention can comprise a plurality of headphones. By means of the respective set of headphones, the kinetosis effect is first detected in the respective passenger and then reduced by initiating acoustic countermeasures. If a passenger has no kinetosis effect, the set of headphones recognizes this via the sensor device and only functions as a playback device of entertainment media.

The sensor device comprises at least one sensor, preferably multiple sensors, which are configured to detect kinetosis effects of the respective passenger. The at least one sensor is arranged or integrated in the first earplug of the set of headphones and is part of the sensor device.

An earplug is to be understood as an element which penetrates at least partially into the auditory canal of the respective passenger. As soon as kinetosis effects are detected in the auditory canal of the respective passenger by means of the sensor device integrated in the earplug, auditory countermeasures are initiated via the two loudspeakers in dependence on the detected kinetosis effects. For example, soothing sounds or music are played back. In particular, binaural tones and/or frequencies are added to the already running music. These acoustic signals result in expanded comfort of the respective passenger, by which kinetosis effects are relieved.

The sensor device preferably comprises at least one first sensor which is configured to detect a body temperature in the auditory canal or at the eardrum of the respective passenger. The respective first sensor is designed as a temperature sensor and detects the body temperature of the wearer either by skin contact or contactlessly, for example optically. Furthermore, the respective first sensor is configured to generate sensor data about the body temperature of the respective passenger. The detection of the body temperature in the auditory canal or on the eardrum is particularly precise. In particular, the sensor device comprises multiple first sensors which are used to detect the body temperature of the respective passenger. Kinetosis effects can be identified via the change of the body temperature of the respective passenger. For example, in the event of an increase of a kinetosis effect, an increase of the body temperature first occurs, wherein the body temperature drops again abruptly from a threshold value.

The sensor device preferably comprises at least one second sensor, which is configured to detect changes in the auditory canal of the respective passenger. Changes in the auditory canal of the respective passenger are to be understood, for example, as pressure variations or length or dimension changes of the auditory canal, which are initiated when the user feels unwell and changes his facial expression. For example, multiple second sensors are provided, wherein these can be designed, for example, as pressure, light, or sound sensors.

According to one preferred embodiment of the invention, the system comprises at least one gyrometer and at least one accelerometer, which are configured to detect forces acting on the respective passenger during the operation of the vehicle. For example, the at least one gyrometer and the at least one accelerometer are integrated in the headphones. In particular, the at least one gyrometer and the at least one accelerometer are part of the sensor device. Alternatively, the at least one gyrometer and the at least one accelerometer are installed in the vehicle and are connected in a signal-transmitting manner to the headphones, for example, in a wired or wireless manner. The at least one gyrometer detects centrifugal forces, thus forces acting transversely to the longitudinal direction of the vehicle, which occur, for example, during cornering. In contrast, the at least one accelerometer detects acceleration forces in and opposite to the longitudinal direction of the vehicle. The forces acting on the respective passenger during the operation of the vehicle are detected, wherein sensor data are generated which enable an acoustic indication of the forces via the loudspeakers of the respective headphones. For example, tones and/or frequencies are added to the music to be played back in real time in such a way that the respective passenger can at least subconsciously adjust to the route of the vehicle, by which relief of the kinetosis effect is achieved in the respective passenger. The added tones and/or frequencies change in dependence on the driving maneuver, in particular in dependence on the longitudinal and lateral forces, so that the perception of the respective passenger accordingly shifts longitudinally and transversely.

The at least one set of headphones preferably comprises a second earplug. Furthermore, the sensor device is preferably also arranged at least partially in the second earplug. In other words, sensors of the sensor device are arranged both in the first and in the second earplug. For example, at least one first sensor for detecting the body temperature in the auditory canal of the respective passenger is arranged in the first earplug, wherein at least one second sensor for detecting changes in the auditory canal of the respective passenger is arranged in the second earplug.

According to one preferred embodiment of the invention, the at least one set of headphones comprises a processing unit which is configured to calculate the kinetosis effect of the respective passenger at least on the basis of sensor data of the sensor device. For example, the processing unit is integrated into the headphones, in particular in an earplug, preferably in an earplug in which no sensors are arranged. If music is played back via the loudspeakers of the respective headphones, noise of the sensor data generated by the sensor device can occur, which can be filtered out by means of the processing unit. A temperature development of the respective set of headphones can also be filtered out by means of the processing unit so as not to corrupt the measurement of the body temperature of the respective passenger.

A more precise detection of the kinetosis effect of the respective passenger is enabled by a combination of first and second sensors. Preferably, all sensor signals may be combined by means of the processing unit, so that the kinetosis of the respective passenger can be calculated from all sensor signals. Sensor data relating to the kinetosis of a respective passenger are therefore detected by the sensor unit and evaluated by the processing unit to determine the well-being of the respective passenger. As soon as the calculation of the kinetosis effect by the processing unit results in a value for the kinetosis effect of the respective passenger which exceeds a threshold value or limiting value of the kinetosis effect for the respective passenger, auditory countermeasures are initiated to reduce the kinetosis effect. This helps the respective passenger to orient himself and adjust to the current driving maneuver of the vehicle, by which the buildup of the kinetosis effect is counteracted.

According to one preferred embodiment of the invention, the respective earplug is configured to close the respective auditory canal of the respective passenger. For example, a seal is arranged on the respective earplug which preferably comes into contact peripherally at the entry of the auditory canal and seals off the auditory canal. Alternatively, the respective earplug is formed essentially complementary to the respective auditory canal and is made elastic in such a way that it preferably comes into contact peripherally at the entry of the auditory canal, in order to seal off the auditory canal. The respective earplug preferably comprises a venting unit. The venting unit is designed to implement venting of the auditory canal in the event of height differences or changes of the auditory canal, so that pressure cannot build up on the eardrum.

According to one preferred embodiment of the invention, the system comprises a control and evaluation unit which is configured to be connected to the at least one set of headphones for data transmission. In particular, the control and evaluation unit is arranged separately from the headphones and is wirelessly connected to the headphones, by which the headphones can be made particularly light and comfortable. Alternatively, the control and evaluation unit can be connected via wire to the headphones. For example, the control and evaluation unit can be designed as an application on a mobile terminal, for example, on a smart phone or tablet. Alternatively, the control and evaluation unit can be implemented in the system of the vehicle or an external computer. In particular, it is also conceivable that the control and evaluation unit is located in a data cloud. The control and evaluation unit can be executed, for example, as a background function so as not to restrict the user in his activities, wherein the processing power for the control and evaluation unit is not coupled to the processing power of the respective set of headphones. Furthermore, music and control data can be transmitted to the loudspeakers of the respective set of headphones via the control and evaluation unit. In particular, the set of headphones also transmits sensor data to the control and evaluation unit. Algorithms are preferably executed on the control and evaluation unit.

Preferably, the control and evaluation unit is configured to detect a health status of the respective passenger via received sensor data relating to the kinetosis effect of the respective passenger. The control and evaluation unit therefore receives sensor data from the sensor device of the respective set of headphones and evaluates them to determine the health status and the well-being of the respective passenger via this. This is advantageous in particular if the passenger is located alone in a vehicle and no help is in the surroundings. For example, the vehicle can be designed as a gondola and can be configured to transport passengers between multiple stations. The control and evaluation unit thus enables the integration of a mobility service to protect the passengers.

The control and evaluation unit is preferably configured to be connected to a display and control system of the vehicle for data transmission. The connection between the control and evaluation unit and the display and control system of the vehicle enables a communication, in particular a transmission of data from the control and evaluation unit to the display and control system of the vehicle. For example, the control and evaluation unit can establish a discomfort of the respective passenger and transmit or indicate this to a driver of the vehicle, for example a bus driver, so that he adjusts his journey accordingly. Alternatively, it is also conceivable that the control and evaluation unit establishes a discomfort of the respective passenger and transmits control commands to an autonomous vehicle to adjust the driving style and to throttle a speed. Therefore, the control and evaluation unit can have an influence via the display and control system of the vehicle on the driving style of the vehicle to reduce kinetosis effects in the respective passenger.

According to a method according to the invention for operating a system according to the invention for reducing a kinetosis effect, the kinetosis effect is detected in the auditory canal of a respective passenger with the aid of a sensor device, which is integrated in at least one first earplug of a set of headphones, wherein acoustic signals for reducing the kinetosis effect of the respective passenger are provided in dependence on the detected kinetosis effect by means of loudspeakers integrated in the headphones.

To detect kinetosis effects in the respective passenger, for example, a body temperature in the auditory canal or at the eardrum of the respective passenger is detected using at least one first sensor of the sensor device. Furthermore, changes in the auditory canal of the respective passenger are preferably detected using at least one second sensor of the sensor device. The changes in the auditory canal of the respective passenger result from changes of a facial expression of the respective passenger and permit the detection of the development of kinetosis effects in the respective passenger. In other words, a discomfort of the respective passenger can thus be detected.

Preferably, forces acting on the respective passenger are detected by means of at least one gyrometer and at least one accelerometer during the operation of the vehicle, wherein the acoustic signals for reducing the kinetosis effect of the respective passenger are also generated in dependence on these forces. In particular, the sensor data of the first and second sensors can be compared to the sensor data of the gyrometer and the accelerometer or related thereto in order to provide monitoring and redundancy of all sensor data.

As soon as kinetosis effects are detected in the auditory canal of the respective passenger by means of the sensor device, auditory countermeasures are initiated via the two loudspeakers in dependence on the detected kinetosis effects and optionally in dependence on the forces acting on the respective passenger. For example, soothing sounds or music are played back. In particular, binaural tones and/or frequencies are added to the music already running. For example, tones and/or frequencies are added to the music to be played back in real time in such a way that the respective passenger can adjust at least subconsciously to the route of the vehicle, by which relief of the kinetosis effect is achieved in the respective passenger. The added tones and/or frequencies change in dependence on the driving maneuver, in particular in dependence on the longitudinal and lateral forces, so that the perception of the respective passenger accordingly shifts longitudinally and transversely.

A threshold value is preferably set by the respective passenger for the detected kinetosis effect of the respective passenger, wherein acoustic signals for reducing the kinetosis effect of the respective passenger are only provided after exceeding the threshold value. In other words, the respective passenger can manually and variably set the sensitivity and the provision of the acoustic signals for reducing the kinetosis effect. In particular, this setting takes place, for example, directly on the set of headphones or via the control and evaluation unit, which is preferably executed as an application on a smart phone.

One preferred exemplary embodiment of the invention is explained in more detail hereinafter on the basis of the two figures, wherein identical or similar elements are provided with the same reference signs. In the figures

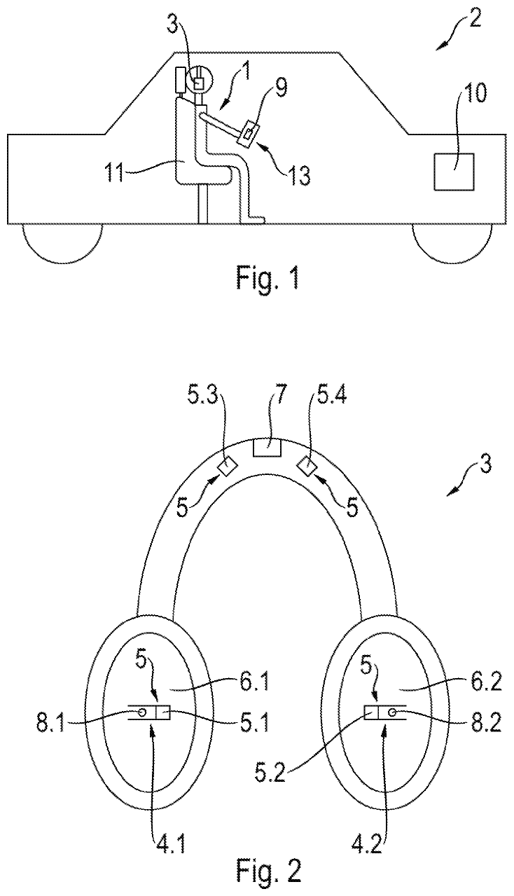

FIG. 1 shows a greatly simplified schematic illustration of a vehicle having a passenger and a system according to the invention for reducing a kinetosis effect in the passenger, and

FIG. 2 shows a greatly simplified schematic illustration of a set of headphones of the system according to the invention according to FIG. 1.

A vehicle 2 is shown in FIG. 1, wherein a passenger 1 is seated on a vehicle seat 11 in a passenger compartment of the vehicle 2 and wears a set of headphones 3. The set of headphones 3 is shown enlarged, but greatly simplified, in FIG. 2. The set of headphones 3 is part of a system according to the invention for reducing a kinetosis effect in the passenger 1. In the present case, the vehicle 2 is designed as an autonomously driving vehicle 2 and is operated via a display and control system 10.

According to FIG. 2, the set of headphones 3 comprises first and second loudspeakers 6.1, 6.2, which are connected to one another via a headband 12 and are configured to press against the ears of the passenger 1 and play back music. Furthermore, the set of headphones 3 has a first and second earplug 4.1, 4.2, which are formed in the present case centrally in the region of the loudspeakers 6.1, 6.2 on the set of headphones 3. A part of a sensor device 5 is integrated in the first and second earplugs 4.1, 4.2, which can detect the kinetosis effect in the auditory canal of the passenger 1, in order to provide acoustic signals for reducing the kinetosis effect via the two loudspeakers 6.1, 6.2 if a threshold value for the kinetosis effect of the passenger 1 is exceeded.

A first sensor 5.1 of the sensor device 5 is arranged in the first earplug 4.1 and detects a body temperature in the auditory canal or at the eardrum of the passenger 1. A second sensor 5.2 of the sensor device 5 is arranged in the second earplug 4.2 and detects changes in the auditory canal of the passenger 1, which result from a change of his facial expression. To carry out a more precise measurement, the respective earplug 4.1, 4.2 is configured to close the respective auditory canal of the passenger 1, wherein the respective earplug 4.1, 4.2 has a venting unit 8.1, 8.2 in order to prevent a pressure buildup in the auditory canal.

Furthermore, the sensor device 5 also comprises a gyrometer 5.3 and an accelerometer 5.4, which are integrated in the set of headphones 3 and detect forces acting on the passenger 1 during the operation of the vehicle 2. Furthermore, the set of headphones 3 has a processing unit 7, which can process all sensor data of the sensor device 5 to calculate the kinetosis effect of the passenger 1 precisely.

A control and evaluation unit 9 is also part of the system according to the invention and is executed as an application on a smart phone 13 of the passenger 1. The control and evaluation unit 9 is configured to be wirelessly connected to the set of headphones 3 for data transmission. Furthermore, the control and evaluation unit 9 is also configured to detect a health status of the passenger 1 via the received sensor data relating to the kinetosis effect of the passenger 1 and to transmit a discomfort to the display and control system 10 of the vehicle 2, in order to adjust the driving style of the vehicle 2 and thus relieve the kinetosis effects in the passenger 1.

The kinetosis effect is thus detected in the auditory canal of the passenger 1 by means of the first and second sensor 5.1, 5.2 of the sensor device 5, wherein acoustic signals for reducing the kinetosis effect are provided via the loudspeakers 6.1, 6.2 in dependence on the detected kinetosis effect and in dependence on sensor data of the gyrometer 5.3 and the accelerometer 5.4. The passenger 1 can set a threshold value for the detected kinetosis effect via an input at the control and evaluation unit 9, wherein the acoustic signals for reducing the kinetosis effect are only provided upon exceeding the threshold value. If the control and evaluation unit 9 detects a disadvantageous development of the kinetosis effects in the passenger 1 upon evaluation of the sensor data, auditory countermeasures are automatically taken in that the loudspeakers 6.1, 6.2 are activated to play back tones and/or frequencies which cause the passenger 1 to feel orientation and an expanded sense of comfort, so that the kinetosis effects are relieved.

### LIST OF REFERENCE SIGNS

1 passenger
2 vehicle
3 headphones
4.1 first earplug
4.2 second earplug
5 sensor device
5.1 first sensor
5.2 second sensor
5.3 gyrometer
5.4 accelerometer
6.1 first loudspeaker
6.2 second loudspeaker
7 processing unit
8.1 first venting unit
8.2 second venting unit
9 control and evaluation unit
10 display and control systems
11 vehicle seat
12 headband
13 smart phone

1. A system for reducing a kinetosis effect in at least one passenger of a vehicle, comprising at least one set of headphones having two loudspeakers and having at least one first earplug, wherein the at least one set of headphones is configured to detect the kinetosis effect in the auditory canal of the respective passenger at least by means of a sensor device integrated in the first earplug and to provide acoustic signals to reduce the kinetosis effect of the respective passenger by means of the two loudspeakers in dependence on the detected kinetosis effect.

2. The system as claimed in claim 1, wherein the sensor device comprises at least one first sensor, which is configured to detect a body temperature in the auditory canal or at the eardrum of the respective passenger.

3. The system as claimed in claim 1, wherein the sensor device comprises at least one second sensor, which is configured to detect changes in the auditory canal of the respective passenger.

4. The system as claimed in claim 1, comprising at least one gyrometer and at least one accelerometer, which are configured to detect forces acting on the respective passenger during the operation of the vehicle.

5. The system as claimed in claim 1, wherein the at least one set of headphones comprises a second earplug.

6. The system as claimed in claim 5, wherein the sensor device is at least partially also arranged in the second earplug.

7. The system as claimed in claim 1, wherein the at least one set of headphones comprises a processing unit, which is configured to calculate the kinetosis effect of the respective passenger at least on the basis of sensor data of the sensor device.

8. The system as claimed in claim 1, wherein the respective earplug is configured to close the respective auditory canal of the respective passenger.

9. The system as claimed in claim 1, wherein the respective earplug comprises a venting unit.

10. The system as claimed in claim 1, comprising a control and evaluation unit, which is configured to be connected to the at least one set of headphones for data transmission.

11. The system as claimed in claim 10, wherein the control and evaluation unit is configured to detect a health status of the respective passenger at least via the received sensor data relating to the kinetosis effect of the respective passenger.

12. The system as claimed in claim 10, wherein the control and evaluation unit is configured to be connected to a display and control system of the vehicle for data transmission.

13. A method for operating a system for reducing a kinetosis effect as claimed in any one of the preceding claims, wherein the kinetosis effect is detected in the auditory canal of a respective passenger by means of a sensor device, which is integrated in at least one first earplug of a set of headphones, wherein acoustic signals for reducing the kinetosis effect of the respective passenger are provided in dependence on the detected kinetosis effect by means of loudspeakers integrated in the headphones.

14. The method as claimed in claim 13, wherein forces acting on the respective passenger during the operation of the vehicle are detected by means of at least one gyrometer and at least one accelerometer, wherein the acoustic signals for reducing the kinetosis effect of the respective passenger are also generated in dependence on these forces.

15. The method as claimed in claim 13, or wherein a threshold value for the detected kinetosis effect of the respective passenger is set by the respective passenger, wherein acoustic signals for reducing the kinetosis effect of the respective passenger are only provided after exceeding the threshold value.
